(19) United States
(12) Patent Application Publication
(10) Pub. No.: US 2015/0305751 A1
(43) Pub. Date: Oct. 29, 2015

(54) Tissue Compression Device with Deflecting Pressure Element

(71) Applicant: Medtronic Vascular, Inc., Santa Rosa, CA (US)

(72) Inventors: Mark Hoff, Windsor, CA (US); Gary Kelly, Windsor, CA (US); Sanaz Saatchi, San Francisco, CA (US); Brian Joseph Mason, Palo Alto, CA (US); John W. Lai, San Bruno, CA (US); Remy O'Leary Pieron, San Francisco, CA (US); Yusuke Miyashita, San Mateo, CA (US)

(73) Assignee: Medtronic Vascular, Inc., Santa Rosa, CA (US)

(21) Appl. No.: 14/262,316

(22) Filed: Apr. 25, 2014

(51) Int. Cl. A61B 17/132; A61B 17/12; A61B 17/135

(52) U.S. Cl. CPC A61B 17/1325; A61B 17/135; A61B 17/12009

(57) ABSTRACT

Tissue compression devices with deflecting pressure elements and methods of using the same are described herein. The tissue compression devices include a pressure element and a retention structure attached to the pressure element. The retention structure may be configured to retain the intermediate portion of the pressure element over selected tissue in a selected location on a patient (such as, e.g., over a radial artery access site). Movement of first and second end portions of the pressure element towards each other along an actuation axis causes the intermediate portion of the pressure element to deflect away from the actuation axis towards the selected tissue.

Tissue compression devices with deflecting pressure elements and methods of using the same are described herein.

### BACKGROUND

The diagnosis and treatment of coronary artery disease is now often accomplished using vascular delivery apparatus and techniques. Vascular delivery may provide a variety of advantages because access to desired locations within a patient's body may be obtained without the need for general anesthetic or more invasive surgical techniques. Access to peripheral arteries may be accomplished using a sheath having a hemostatic valve that is inserted into the peripheral artery. A catheter or other device can then be introduced into the vasculature of the patient through that sheath to the desired location within the vasculature.

Access for these percutaneous coronary procedures may be obtained through a radial artery of the patient. Access through a radial artery may provide a number of advantages including improved patient mobilization and reduced cost. The use of a distal radial artery may, for example, allow for compression to be directly applied to the artery to achieve and maintain hemostasis.

One potential complication with any arterial access is, however, achieving hemostasis during and/or after a procedure. Arterial blood flow is pulsatile in nature and may present challenges to any practitioner seeking to achieve hemostasis at an arterial access site. Upon completion of a procedure and after removal of a catheter or other device located in the access site, pressure may be applied to the access site to achieve hemostasis and close the access site. Applying pressure at or at a point slightly upstream of the access site is one technique that may be used for achieving hemostasis. In many instances, continuous pressure may be needed to achieve hemostasis at the access site. Although it may be advantageous for the pressure to remain constant, in some instances a reduction in the level of applied pressure may be advantageous after an initially higher level of pressure is applied to the access site. Gradual reduction in the compression pressure may allow blood to flow through the artery of the access site to allow blood to reach tissue downstream from the access site. That blood flowing through the artery can, in some instances, improve clotting to achieve hemostasis without continuing application of compression.

### SUMMARY

Tissue compression devices with deflecting pressure elements and methods of using the same are described herein.

In one or more embodiments, the tissue compression devices described herein include a pressure element and a retention structure attached to the pressure element. The retention structure may, in one or more embodiments, be configured to retain the intermediate portion of the pressure element over selected tissue in a selected location on a patient as described herein. Movement of first and second end portions of the pressure element towards each other along an actuation axis causes, in one or more embodiments, the intermediate portion of the pressure element to deflect away from the actuation axis towards the selected tissue.

The phrase “towards each other” is used to describe a reduction in the distance between the first and second end portions of the pressure element to cause deflection of the intermediate portion of the pressure element. As used herein, the phrase “towards each other” (and variations thereof) does not require movement of both the first and second end portions of the pressure element, i.e., the position of one end portion may be held constant while the other end portion is moved or both end portions may be moved—either variation results in movement of the end portions “towards each other” as used in connection with the tissue compression devices described herein.

As used herein, the phrase “over selected tissue” (and variations thereof) means that the intermediate portions of the pressure elements of the tissue compression devices are positioned to move towards the selected tissue—the term “over” is, therefore, used only in a relative sense and the intermediate portion may be located above or below the selected tissue in absolute terms with respect to the direction of gravitational forces.

In one or more embodiments of the tissue compression devices described herein, an actuator is attached to the first and second end portions of the pressure element, the actuator being configured to move the first and second end portions of the pressure element towards each other when actuated by a user.

The tissue compression devices and methods described herein may be used to apply pressure to selected tissue at any selected location on a patient using deflection of the intermediate portion of the pressure element, but the tissue compression devices may be particularly well-suited to apply pressure to tissue that includes a radial artery or other blood vessel that may, in one or more embodiments, be used as an access site for a percutaneous procedure. Applying pressure at an access site may provide control over bleeding at that location while still allowing blood flow through the radial artery.

In a first aspect, one or more embodiments of the tissue compression devices described herein may include: a pressure element comprising a first end portion, a second end portion, and an intermediate portion located between the first and second end portions; and retention structure attached to the pressure element, wherein the retention structure is configured to retain the intermediate portion of the pressure element over selected tissue in a selected location; wherein the pressure element is configured such that movement of the first and second end portions of the pressure element towards each other along an actuation axis causes the intermediate portion of the pressure element to deflect away from the actuation axis towards the selected tissue.

In one or more embodiments, the tissue compression devices of the first aspect described herein may include an actuator attached to the first and second end portions of the pressure element, wherein the actuator is configured to move the first and second end portions of the pressure element towards each other. In one or more embodiments, the actuator comprises a threaded rod located along the actuation axis and connected to the first and second end portions of the pressure element in a manner configured to move the first and second end portions of the pressure element towards each other when the threaded rod is rotated about the actuation axis. In one or more embodiments, the actuator comprises a ratcheting device attached to the first and second end portions of the pressure element.

In one or more embodiments of the tissue compression devices of the first aspect described herein, the pressure element is configured such that deflection of the intermediate portion away from the actuation axis increases as the first and second end portions are moved closer to each other along the actuation axis, and wherein deflection of the intermediate portion away from the actuation axis decreases as the first and second end portions are moved away from each other along the actuation axis after having been moved closer to each other along the actuation axis.

In one or more embodiments of the tissue compression devices of the first aspect described herein, the intermediate portion of the pressure element comprises a substantially flat surface facing away from the actuation axis.

In one or more embodiments of the tissue compression devices of the first aspect described herein, the pressure element further comprises a conformable pad positioned over the selected tissue. The conformable pad comprises a fluid-filled bladder.

In one or more embodiments of the tissue compression devices of the first aspect described herein, the intermediate portion of the pressure element is configured to allow visual access to the selected tissue when the intermediate portion contacts the selected tissue.

In a second aspect, one or more embodiments of the methods described herein may include: positioning the intermediate portion of a pressure element of a tissue compression device over selected tissue at a selection location on a patient, wherein the intermediate portion of the pressure element is located between a first end portion and a second end portion of the pressure element; and moving the first and second end portions of the pressure element closer together along an actuation axis while retaining the intermediate portion of the pressure element over the selected tissue, wherein the intermediate portion applies increasing pressure on the selected tissue when the first and second end portions of the pressure element are moved closer to each other, and wherein the increasing pressure is directed away from the actuation axis.

In one or more embodiments of the methods of the second aspect described herein, the selected tissue is located on a limb of a patient, and wherein the tissue compression device encircles the limb, and further wherein the actuation axis is transverse to a limb axis extending along a length of the limb.

In one or more embodiments of the methods of the second aspect described herein, the tissue compression device comprises an actuator that comprises a threaded rod attached to the first and second end portions of the pressure element, wherein the threaded rod is located along the actuation axis, wherein moving the first and second end portions of the pressure element closer together comprises rotating the threaded rod in a first direction about the actuation axis. In one or more embodiments, moving the first and second end portions of the pressure element away from each other by rotating the threaded rod in a second direction about the actuation axis.

In one or more embodiments of the methods of the second aspect described herein, the method further comprises attaching the tissue compression device to a patient such that the intermediate portion of the pressure element is positioned over the selected tissue, wherein the attaching comprises: attaching a first end of a retention structure to the first end portion of the pressure element; wrapping the retention structure around a limb on which the selected tissue is located; and attaching a second end of the retention structure to the second end portion of the pressure element.

In a third aspect, one or more embodiments of the tissue compression devices described herein may include: a pressure element comprising a first end portion, a second end portion, and an intermediate portion located between the first and second end portions; and retention structure attached to the pressure element, wherein the retention structure is configured to retain an intermediate portion of the pressure element over selected tissue in a selected location on a limb by forming a closed loop encircling the limb, wherein the encircled limb defines a limb axis extending through the closed loop; wherein the pressure element is configured such that the intermediate portion of the pressure element deflects in a first direction towards the limb as the first and second end portions of the pressure element move towards each other in a second direction, wherein the first direction is transverse to both the second direction and the limb axis.

In one or more embodiments of tissue compression devices of the third aspect described herein, the tissue compression device may include an actuator attached to the first and second end portions of the pressure element, wherein the actuator is configured to move the first and second end portions of the pressure element towards each other in the second direction. In one or more embodiments, the actuator comprises a threaded rod aligned with the second direction and connected to the first and second end portions of the pressure element in a manner configured to move the first and second end portions of the pressure element towards each other when the threaded rod is rotated about an actuation axis extending along a length of the threaded rod. In one or more embodiments, the actuator comprises a ratcheting device attached to the first and second end portions of the pressure element.

In one or more embodiments of tissue compression devices of the third aspect described herein, the pressure element is configured such that deflection of the intermediate portion in the first direction towards the limb increases as the first and second end portions are moved closer to each other along the second direction, and wherein deflection of the intermediate portion in the first direction decreases as the first and second end portions are moved away from each after having been moved closer to each other along the second direction.

In one or more embodiments of tissue compression devices of the third aspect described herein, the intermediate portion of the pressure element comprises a substantially flat surface over the selected tissue in the selected location on the limb when the tissue compression device encircles the limb.

In one or more embodiments of tissue compression devices of the third aspect described herein, the pressure element further comprises a conformable pad positioned over the selected tissue. In one or more embodiments, the conformable pad comprises a fluid-filled bladder.

In one or more embodiments of tissue compression devices of the third aspect described herein, the intermediate portion of the pressure element is configured to allow visual access to the selected tissue when the intermediate portion is positioned over the selected tissue.

In a fourth aspect, one or more of the methods described herein may include: positioning the intermediate portion of a pressure element of a tissue compression device over selected tissue on a limb such that the tissue compression device encircles the limb and wherein the limb comprises a limb axis that extends through an opening formed by the tissue compression device, wherein the intermediate portion of the pressure element is located between a first end portion and a second end portion of the pressure element; and moving the first and second end portions of the pressure element closer together along a first direction while retaining the intermediate portion of the pressure element over the selected tissue, wherein the intermediate portion moves in a second direction to apply increasing pressure on the selected tissue when the first and second end portions of the pressure element are moved closer to each other, and wherein the first direction is transverse to both the second direction and the limb axis.

In one or more embodiments of methods of the fourth aspect described herein, the tissue compression device comprises an actuator that comprises a threaded rod attached to the first and second end portions of the pressure element, wherein the threaded rod is aligned with the first direction, wherein moving the first and second end portions of the pressure element closer together along the first direction comprises rotating the threaded rod about the actuation axis.

As used herein and in the appended claims, the singular forms “a,” “an,” and “the” include plural referents unless the context clearly dictates otherwise. Thus, for example, reference to “a” or “the” component may include one or more of the components and equivalents thereof known to those skilled in the art. Further, the term “and/or” means one or all of the listed elements or a combination of any two or more of the listed elements.

It is noted that the term “comprises” and variations thereof do not have a limiting meaning where these terms appear in the accompanying description. Moreover, “a,” “an,” “the,” “at least one,” and “one or more” are used interchangeably herein.

The above summary is not intended to describe each embodiment or every implementation of the tissue compression devices or methods described herein. Rather, a more complete understanding of the invention will become apparent and appreciated by reference to the following Description of Illustrative Embodiments and claims in view of the accompanying figures of the drawing.

### BRIEF DESCRIPTION OF THE VIEWS OF THE DRAWING

FIG. 1 is a schematic side view of one embodiment of a tissue compression device.

FIG. 2A is a perspective view of the pressure element of the tissue compression device of FIG. 1 in isolation (i.e., removed from the tissue compression device).

FIGS. 2B-2C are side views that illustrate operation of the isolated pressure element depicted in FIG. 2A.

FIG. 3 is a schematic view of the tissue compression device of FIG. 1 located on a limb (the outline of which is depicted in FIG. 3).

FIG. 4 is a schematic view of the tissue compression device of FIG. 3 after moving the end portions of the pressure element towards each other as described herein.

FIG. 5 is a perspective view of another illustrative embodiment of a tissue compression device as described herein.

FIG. 6 is a side view of the tissue compression device of FIG. 5.

FIG. 7 is an enlarged perspective view of a portion of the tissue compression device depicted in FIGS. 5 and 6.

### DESCRIPTION OF ILLUSTRATIVE EMBODIMENTS

In the following description of illustrative embodiments, reference is made to the accompanying figures of the drawing which form a part hereof, and in which are shown, by way of illustration, specific embodiments. It is to be understood that other embodiments may be utilized and structural changes may be made without departing from the scope of the present disclosure.

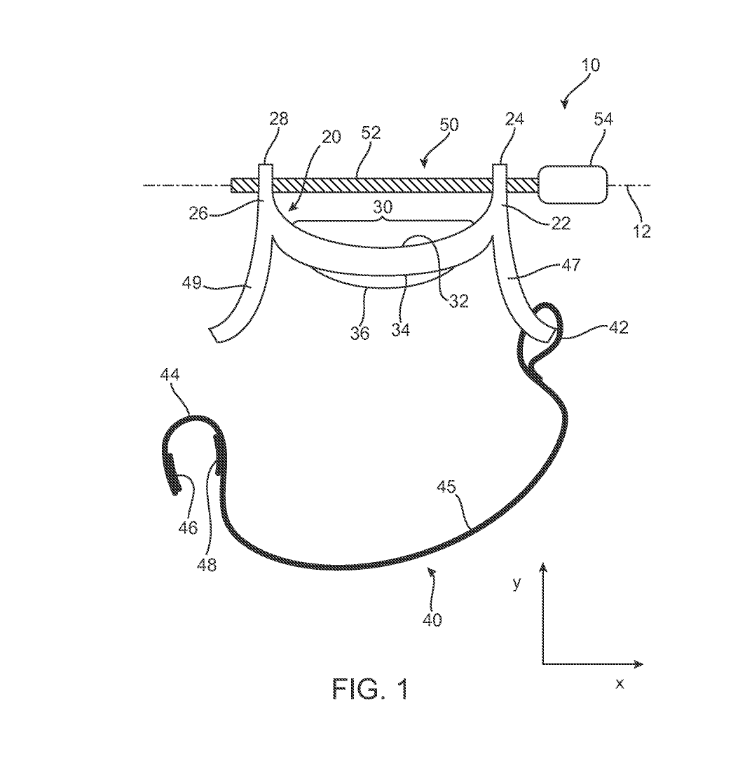

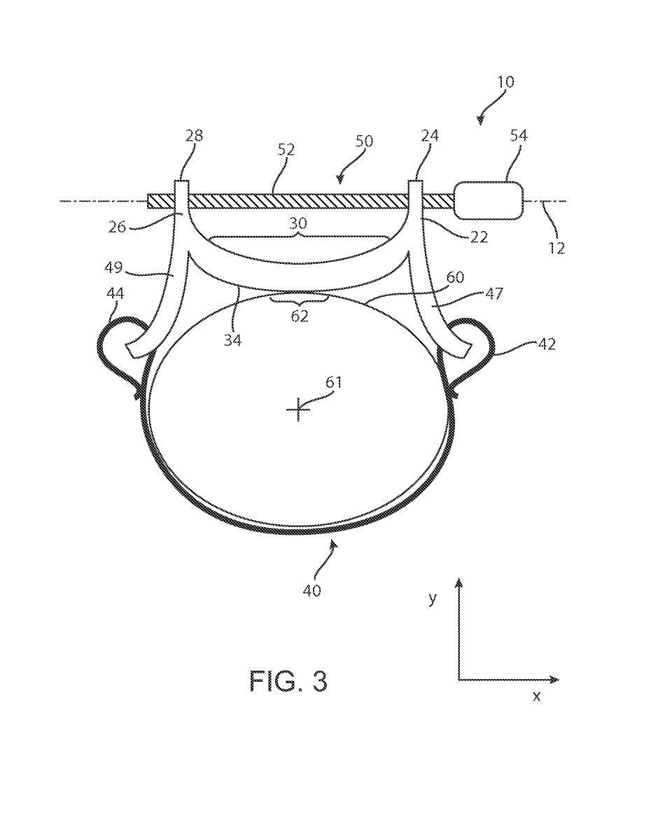

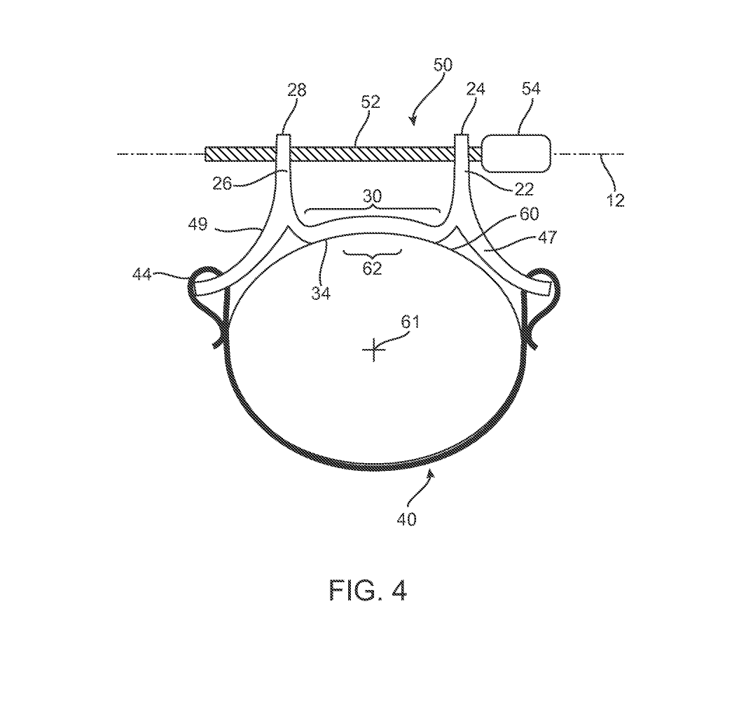

FIG. 1 is a schematic side view of one illustrative embodiment of a tissue compression device 10, while FIGS. 3 and 4 depict the tissue compression device 10 on a limb. The tissue compression device 10 includes a pressure element 20 and a retention structure 40 attached to the pressure element 20 and configured to retain an intermediate portion 30 of the pressure element 20 over selected tissue in a selected location on a patient as described herein (e.g., on a limb such as an arm or leg).

In one or more embodiments, the pressure element 20 includes a first end portion 22 and a second end portion 26. The first end portion 22 includes the first end 24 of the pressure element 20, and the second end portion 26 includes the second end 28. An intermediate portion 30 is located between the first end portion 22 and the second end portion 26. The intermediate portion 30 of the pressure element 20 includes a top surface 32 and a bottom surface 34. The bottom surface 34 of the pressure element 20 faces the selected tissue over which the intermediate portion 30 is located when the tissue compression device 10 is in use.

The depicted illustrative embodiment of the retention structure 40 includes a strap 45 along with a first end connector 47 attached to the first end portion 22 of the pressure element 20 and a second end connector 49 attached to the second end portion 26 of the pressure element 20. The strap 45 extends from the first end connector 47 to the second end connector 49 when the tissue compression device 10 is in use.

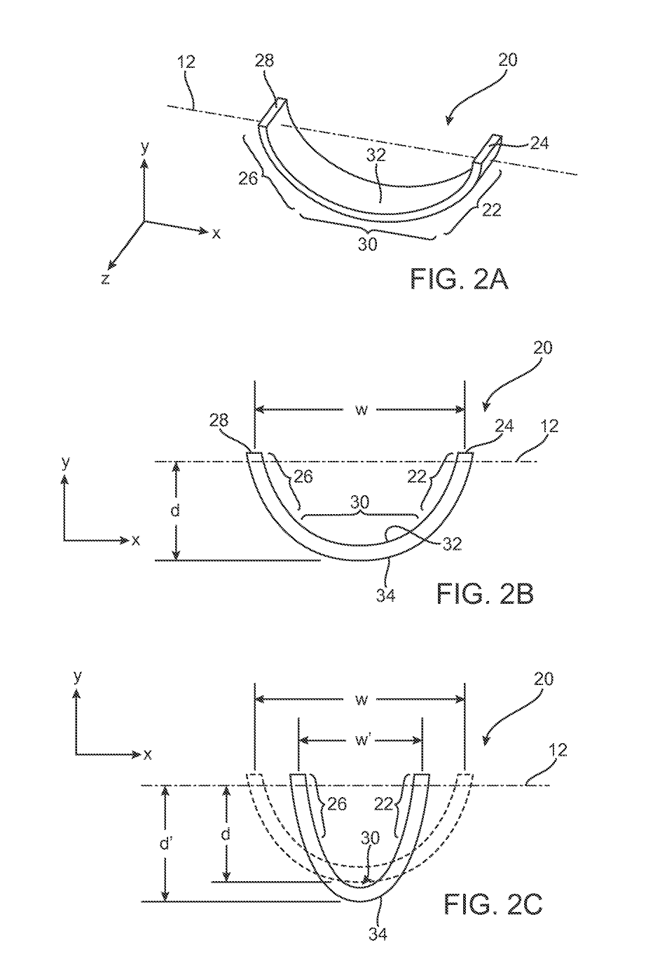

The illustrative embodiment of pressure element 20 used in the tissue compression device 10 is depicted in isolation (i.e., removed from the tissue compression device 10) in FIGS. 2A-2C to provide an opportunity to describe the principles on which the pressure elements of the tissue compression devices described herein operate. The pressure element 20 includes a first end portion 22 that includes the first end 24 and a second end portion 26 including the second end 28, with an intermediate portion 30 located between the first and second end portions 22 and 26. An actuation axis 12 extends through the first and second end portions 22 and 26.

The pressure element 20 may, in one or more embodiments, be described generally as being C-shaped in which the upper surface 32 faces the actuation axis 12. In one or more embodiments, the upper surface 32 can be described as forming a generally concave shape with respect to the actuation axis 12. The pressure element 20 also includes a bottom surface 34 that faces away from the actuation axis 12 can be described as forming a generally convex shape with respect to the actuation axis 12.

As described herein, pressure elements used in tissue compression devices may be configured such that movement of the first and second end portions 22 and 26 towards each other along an actuation axis 12 causes the intermediate portion 30 of the pressure element 20 to deflect away from the actuation axis. This operating principle is depicted in FIGS. 2B and 2C, with reference to actuation axis 12 extending through the first and second end portions 22 and 26.

As depicted in the side view of FIG. 2B, the pressure element 20 has a normal or default width w between its first and second end portions 22 and 26. Furthermore, the pressure element 20 has a normal or default depth d measured from actuation axis 12 to the bottom surface 34 in the intermediate portion 30 of the pressure element 20. As the first and second end portions 22 and 26 are moved towards each other along actuation axis 12 (reducing width w), the intermediate portion 30 of the pressure element 20 is deflected away from the actuation axis 12 (increasing depth d).

Exemplary changes in the width and depth are depicted in FIG. 2C in which the pressure element 20 is also seen in its default or normal shape in broken lines. As the first and second end portions 22 and 26 are moved together along actuation axis 12 to a width w′ (where width w′ is less than width w), the normal or default depth d increases to a depth of d′ as depicted in FIG. 2C (i.e., depth d′ is greater than depth d).

Deflection of the intermediate portion 30 of the pressure element 20 away from the actuation axis 12 can, in one or more embodiments, be described as deflection in a Y axis direction where the actuation axis 12 defines an orthogonal X axis (as seen in the reference axes included in FIGS. 1 and 2A-2C). In terms of the reference axes, movement of the first and second end portions 22, 26 of the pressure element 20 towards each other may, in one or more embodiments, cause the intermediate portion 30 of the pressure element 20 to deflect away from the actuation axis 12 in a direction that is substantially parallel to the Y axis.

As described herein, the pressure element 20 may have a default or normal shape when the pressure element 20 is not subjected to any external force (other than gravity) and, further, the deflection of the intermediate portion 30 caused by movement of the first and second end portions 22, 26 towards each other may be elastic deflection. By elastic deflection, we mean that the pressure element 20 will substantially return to its default or normal shape over a relatively short period of time (e.g., 5 minutes, 2 minutes, or even 1 minute or less) in the absence of external forces that cause the intermediate portion 30 of the pressure element 20 to deflect as described herein. In one or more embodiments, however, the pressure element 20 may experience some permanent deformation as a result of the deflection process, but such permanent deformation may be limited to, e.g., 20%, 10% or even 5% or less of the total deflection caused in the intermediate portion 30 under normal use of the tissue compression devices described herein. In a pressure element 20 providing elastic deflection of the intermediate portion 30, increases in deflection of the intermediate portion 30 may be, in one or more embodiments, reversed by moving the first and second end portions 22, 26 of the pressure element 20 away from each other after those end portions 22, 26 have been moved towards each other. Reversing the deflection of the intermediate portion 30 will typically cause a corresponding decrease in pressure applied to selected tissue by the pressure element 20.

Pressure elements used in tissue compression devices as described herein, such as, e.g., pressure element 20, can be constructed of any suitable material or combination of materials, e.g., polymeric, metallic, etc. that provide a pressure element with the deflection characteristics as described herein. In one or more embodiments, the pressure element 20 can include substantially transparent material such that a user has visual access to the selected tissue over which the intermediate portion 30 is located (e.g., a percutaneous access site, etc.).

Although the illustrative embodiment of pressure element 20 has a generally uniform thickness and width, in one or more alternative embodiments of pressure elements that may be used in tissue compression devices as described herein, the pressure elements may have non-uniform thicknesses and/or widths. In another variation, the tissue compression devices described herein may include a pressure element in the form of a unitary structure such as, e.g., pressure element 20. In one or more alternative embodiments, however, the pressure element used in a tissue compression device as described herein may be a composite structure that includes multiple components arranged to provide the deflection and resulting tissue compression as described herein.

In one or more embodiments of the tissue compression devices described herein, a pad 36 as depicted in FIG. 1 may be located on the bottom surface 34 in the intermediate portion 30 of the pressure element 20. In one or more embodiments, the pad 36 may be a conformable pad that is capable of deforming to conform to the shape of a surface against which it is compressed. In one or more embodiments, the conformable pad 36 may include a variety of one or more resilient, conformable materials, e.g., gels, foams, etc. In one or more alternative embodiments, the conformable pad 36 may be in the form of a fluid-filled bladder where the fluid may include one or more liquids and/or gases. In one or more embodiments in which the conformable pad 36 is in the form of a fluid-filled bladder, the volume of fluid in the bladder may be adjusted (i.e., increased and/or decreased) using, e.g., a syringe, pump, etc. In one or more embodiments, the conformable pad 36 may be transparent. If, for example, the pressure element 20 is also transparent within the intermediate portion 30 to allow visual access to tissue, over which the intermediate portion 30 is located, through both the pressure element 20 and the conformable pad 36.

In the illustrative embodiment depicted in FIG. 1, the retention structure 40 is in the form of a strap 45 and connectors 47 and 49 that may be configured to, in one or more embodiments, wrap around a limb that includes the selected tissue (e.g., a radial artery) to be compressed by the pressure element 20 of the tissue compression device 10. Further, in one or more embodiments, the retention structure 40 and the pressure element 20 may be described as encircling a limb when the pressure element 20 is retained on the limb by the retention structure 40.

The retention structure 40 includes, in one or more embodiments, a first connector 47 attached to the first end portion 22 of the pressure element 20 and a second connector 49 attached to the second end portion 26 of the pressure element 20. The strap 45 includes, in the depicted illustrative embodiment, a first end 42 attached to the first connector 47 and an opposite free end 44 configured to attach to the second connector 49. The retention structure 40 can also include, in one or more embodiments, fasteners 46 and 48 (e.g., hook and loop fasteners, interlocking mechanical fasteners, etc.) that are configured to allow the strap 45 of the retention structure 40 to be attached to a slot (not shown) or other opening in the second connector 49 in the manner of, e.g., a watch band, bracelet, etc. The depicted form of retention structure 40 is only one example of many different straps, bands, belts, etc. that could be used to secure the pressure element 20 in a selected location on a limb, and the depicted fasteners 46 and 48 are only one form of fasteners that could be used to secure the free end of a strap 45 (alternatives including, e.g., adhesive materials, cohesive materials, buckles, snaps, etc.).

The retention structure 40 can be attached to the pressure element 20 in any suitable manner. For example, in some embodiments, the first and second connectors 47 and 49 can be integrally formed with the first and second end portions 22 and 26 of the pressure element 20. For example, in one or more embodiments, the pressure element 20 and connectors 47 and 49 can be formed from, e.g., metallic and/or polymeric materials that are molded or otherwise formed together to form a unitary article. For example, a pressure element 20 and the connectors 47 and 49 may be formed from a continuous strip of spring steel (or any other suitable material) to provide a pressure element 20 and connectors 47 and 49 having the shape depicted in, e.g., FIG. 1. In one or more alternative embodiments, one or both of the connectors 47 and 49 may be formed as separate components that are attached to the first and/or second end portions 22 and 26 of the pressure element 20. In such an embodiment, one or both of the connectors 47 and 49 may be attached to the pressure element 20 using any suitable technique or combination of techniques, e.g., mechanical fasteners, adhesives, welding, clamps, etc.

Further, the first and second connectors 47 and 49 used to attach a strap 45 of retention structure 40 to the pressure element 20 as described herein can take any suitable shape. For example, in the illustrative embodiment depicted in FIG. 1, the first and second connectors 47 and 49 are each curved such that their respective surfaces facing the intermediate portion 30 of the pressure sensitive element 20 are convex. In such an embodiment, the first and second connectors 47 and 49 curve or arc away from a limb on which the tissue compression device 10 is located. The convex shape of the first and second connectors 47 and 49 may, in one or more embodiments, allow the first and second connectors 47 and 49 to comfortably contact a limb and/or move along the surface of the limb during use of the tissue compression device 10.

The components of the retention structure 40 can be constructed of any suitable material or materials, e.g., polymeric, elastic, leather, natural or synthetic fiber, metallic, etc. Furthermore, the shape of the various components of the retention structure 40 can also vary from those seen in the illustrative embodiments described herein.

In one or more embodiments of the tissue compression devices described herein, an actuator 50 may also be included as a component of the tissue compression device 10. The actuator 50 may, in one or more embodiments, be attached to the first end portion 22 and the second end portion 26 of the pressure element 20. The actuator 50 can take many different forms, so long as the actuator 50 can be used to move the first and second end portions 22, 26 towards each other along the actuation axis 12 to cause deflection of the intermediate portion 30 as described herein. Examples of some potentially useful actuators may include, but are not limited to, clamps, ties, wire, string, springs, etc.

In the illustrative embodiment depicted in FIGS. 1, 3, and 4, the actuator 50 is in the form of a threaded rod that includes threads 52 and is connected to the pressure element 20 through a threaded opening (not shown) in one or both of the first and second portions 22, 26 of the pressure element 20. The threaded actuator 50 also includes a knob 54 that can be used to rotate the actuator 50 about the actuation axis 12. Although the knob 54 is depicted at one end of the actuator 50, it could, in one or more alternative embodiments, be located anywhere along the length of the actuator 50 as appropriate. In one or more embodiments, the actuator 50 can be rotated about the actuation axis 12 in either a clockwise or counterclockwise direction to move the first and second end portions 22, 26 of the pressure element 20 towards and/or away from each other as described herein.

In one or more embodiments, the threaded actuator 50 may be configured to draw or move the second end portion 26 of the pressure element 20 toward the first end portion 22 (e.g., the positive x direction) as the actuator 50 is rotated about actuation axis 12. A collar or collet (not shown) may, in one or more embodiments, be positioned between the first end portion 22 and the knob 54 of the threaded actuator 50 such that the first end portion 22 remains fixed in position relative to the second end portion 26 of the pressure element 20 as the second end portion 26 is drawn or moved closer to the first end portion 22.

In one or more embodiments, the threaded actuator 50 can include a first portion having threads formed in one direction, e.g., clockwise, and a second portion having threads formed in the opposite direction, e.g., counterclockwise. In such embodiments, the first portion of the threaded rod 52 can extend through a threaded opening in the first end portion 22 of the pressure element 20, and the second portion of the threaded actuator 50 can extend through a threaded opening in the second end portion 26 of the pressure element 20. In such an embodiment, rotation of the threaded actuator 50 about the actuation axis 12 moves the first end portion 22 of the pressure element 20 toward the second end portion 26 and also moves the second end portion 26 toward the first end portion 22. Such an arrangement is not, however, required.

As described herein, moving the first and second end portions 22, 26 towards each other along the actuation axis 12 causes intermediate portion 30 of the pressure element 20 between the first and second end portions 22, 26 to move or deflect away from the actuation axis 12 as described herein. It is that deflection or movement of the intermediate portion 30 of the pressure element 20 that provides tissue compression of the selected tissue over which the intermediate portion 30 is located.

In one or more embodiments, the selected tissue to be compressed, i.e., the selected tissue over which the intermediate portions of the tissue compression devices described herein are located, may be found on a limb, e.g., an arm, leg, etc. In one or more embodiments, the tissue compression devices can be located on any suitable limb to compress selected tissue located on that limb. FIG. 3 is a schematic view of the tissue compression device 10 of FIG. 1 located on a limb 60 (the optional conformable pad 36 is removed in this view in the interest of clarity). As described herein and shown in FIG. 3, the retention structure 40 and attached pressure element 20 are configured to retain the intermediate portion 30 of the pressure element 20 over the selected tissue 62 on a limb 60 by encircling the limb.

The encircled limb 60 defines a limb axis 61 extending through the closed loop formed by the pressure element 20 and the retention structure 40. In the view depicted in FIG. 3, the limb axis 61 is transverse to the plane of the figure, i.e., the limb axis 61 may, in one or more embodiments, be described as transverse to both the X axis and the Y axis (thus, the limb axis is along a Z axis). In another manner of characterizing the limb axis 61, it may be described as running from the wrist to the elbow where the limb is the forearm of a patient. As used herein, “transverse” includes both perpendicular and substantially perpendicular arrangements of axes and/or components.

As shown in FIG. 3, the pressure element 20 is configured such that the intermediate portion 30 of the pressure element 20 deflects towards the limb 60 as the first and second end portions 22, 26 are moved towards each other. In one or more embodiments, the pressure element 20 may be described as being configured such that the intermediate portion 30 of the pressure element 20 deflects in a first direction (e.g., along the Y axis) towards the limb 60 as the first and second end portions 22, 26 of the pressure element 20 move towards each other along a second direction (e.g., along the X axis), wherein the first direction is transverse to both the second direction and the limb axis 61 (which is, as described herein, the limb axis is generally aligned along a Z axis that is transverse to both the X and Y axes).

In one or more embodiments of the tissue compression devices described herein, the pressure element 20 can be constructed such that contact area between the intermediate portion 30 of the pressure element 20 and the limb 60 increases as the intermediate portion 30 acts against selected tissue to apply pressure while being deflected away from the actuation axis 12 as described herein. One illustrative embodiment of this principle is depicted in FIG. 4, where the tissue compression device 10 is depicted after deflection of the intermediate portion 30 of the pressure element 20 as a result of movement of the first and second end portions 22, 26 towards each other as described herein. As in FIG. 3, the optional conformable pad 36 has been removed from the tissue compression device 10 as depicted in FIG. 4 for clarity.

As illustrated in FIG. 4, more of the intermediate portion 30 of the pressure element 20 contacts the limb 60 as the intermediate portion 30 deflects away from the actuation axis 12 and towards the selected tissue 62 on limb 60. In one or more embodiments, the intermediate portion 30 of the pressure element 20 may be constructed of a material or materials such that the intermediate portion 30 at least partially takes the shape or conforms to the shape of the selected tissue over which the intermediate portion 30 is located and towards which the intermediate portion 30 is deflected during use of the tissue compression device 10 (the result of which may include applying pressure to a greater surface area of the limb 60).

These characteristics can be adjusted such that, for example, a larger or smaller portion of the intermediate portion 30 of the pressure element 20 can contact the selected tissue in response to deflection of the intermediate portion 30 as described herein. Although not depicted, in one or more embodiments, the tissue in limb 60 may also be deformed or conform (at least partially) to the shape of the intermediate portion 30 of the pressure element 20.

As discussed herein, the pressure element 20 (including intermediate portion 30) can be constructed of one or more different materials that have different elastic properties and those different elastic properties can be, in one or more embodiments, used to control deflection of the intermediate portion 30. For example, first and second end portions 22, 26 can be of a different material from intermediate portion 30. In another example, first and second connectors 47, 49 can be of a different material from first and second end portions 22, 26. Alternatively or in addition to the selection of materials, the shape and/or physical structure (e.g., width, thickness, etc.) of the pressure element 20 may be selected to provide different deflection characteristics and/or to control the degree to which the intermediate portion 30 of the pressure element 20 conforms to the shape of the selected tissue being compressed by the tissue compression device 10.

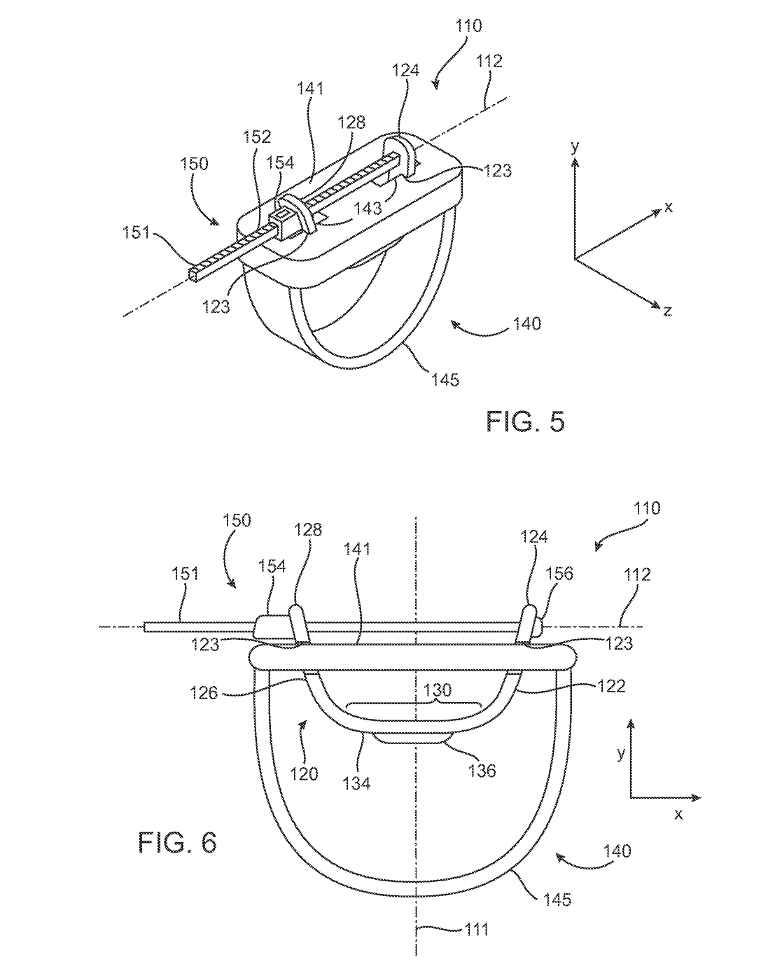

One illustrative embodiment of a tissue compression device 10 has been described in connection with FIGS. 1, 3, and 4. Another alternative illustrative embodiment of a tissue compression device 110 is depicted in connection with FIGS. 5-7.

The illustrative embodiment of tissue compression device 110 includes a pressure element 120 and retention structure 140, along with optional actuator 150. The first end portion 122 of the pressure element 120 terminates at first end 124 and the second end portion 126 of the pressure element 120 ends at second end 128. Intermediate portion 130 of the pressure element 120 is located between the first end portion 122 and the second end portion 126. In the tissue compression device 110, movement of the first and second end portions 122 and 126 towards each other along actuation axis 112 (e.g., the X axis) causes the intermediate portion 130 of the pressure element 120 to deflect away from the actuation axis 112 along the deflection axis 111 (e.g., the Y axis) in a manner similar to that described above in connection with pressure element 20. The pressure element 120 also includes an optional conformable pad 136 located on the intermediate portion 130 of the pressure element 120.

Among the differences between tissue compression device 110 and tissue compression device 10 depicted in FIGS. 1, 3, and 4 is the retention platform 141 provided as a part of the retention structure 140 of tissue compression device 110. In particular, the retention structure 140 includes a strap 145 which is attached to the retention platform 141 and configured to, e.g., encircle a limb or other structures such that a selected tissue can be compressed by the pressure element 120.

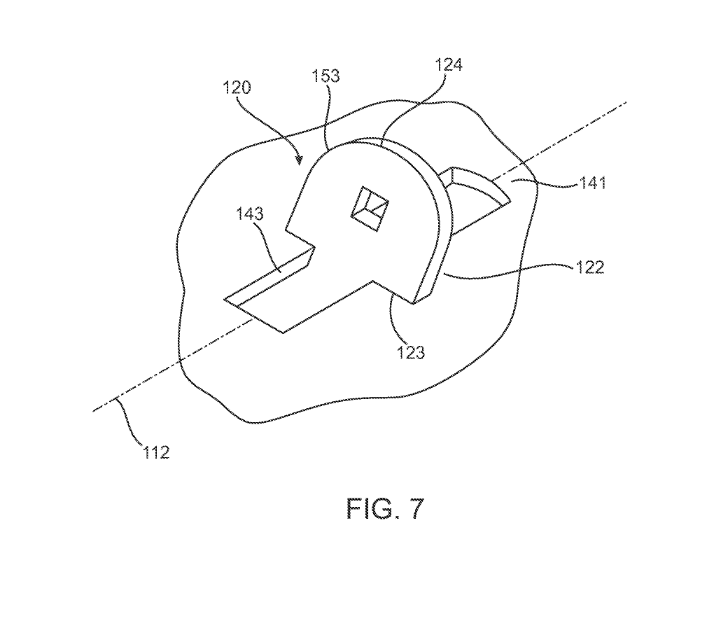

The retention platform 141 also interfaces with the pressure element 120 to retain the pressure element 120 in position over selected tissue on a patient. In particular, the retention platform 141 includes slots 143 that, together with slots 123 formed in pressure element 120, act together to retain the pressure element 120 in position with respect to the retention platform 141. The interface between one of the slots 143 in retention platform 141 and one of the slots 123 in the pressure element 120 is, perhaps, best seen in FIG. 7. The cooperating slot arrangement depicted in FIG. 7 retains the pressure element 120 in position with respect to the retention platform 141 while also allowing movement of the first and second end portions 122 and 126 of the pressure element 120 along the actuation axis 112 such that the pressure element 120 can operate in a manner as described herein.

Although both the first and second end portions 122 and 126 are attached to the platform 141 using cooperating slots 123 and 143 as described herein, in one or more alternative embodiments only one of the end portions may need to be attached to the platform 141 in a manner that allows movement of the end portion. Furthermore, many other structures and/or mechanisms could be used to allow movement of one or both of the end portions of a pressure element used in a tissue compression device as described herein.

Another difference between tissue compression device 110 and tissue compression device 10 depicted in FIGS. 1, 3, and 4 is found in the actuator 150 of the tissue compression device 110. In particular, the actuator 150 is in the form of a ratcheting device which includes a ladder or slot structure 152 formed in an elongated body 151 and a complementary pawl 154 that interacts with the ladder or slot structure 152 in a manner similar to that found in, e.g., a wire tie, zip tie, ratcheting buckle assembly, etc. Such ratcheting devices typically allow for movement in one direction and resist movement in the opposite direction along the elongated body 151. As used in tissue compression device 110, the elongated body 151 of the actuator 150 extends through openings 153 (see, e.g., FIG. 7) in one or both end portions 122 and/or 126 of the pressure element 120 and includes, in one or more embodiments, a stop 156 at one end to prevent the elongated body 151 from passing through the opening 153 in the first end portion 122 of the pressure element 120.

Movement of the first and second end portions 122 and 126 of the pressure element 120 towards each other is, in the depicted embodiment of tissue compression device 110, accomplished by moving pawl 154 of actuator 150 towards the stop 156 at the end of the elongated body 151. As the pawl 154 moves towards the stop 156, the first and second end portions 122 and 126 of the pressure element 120 are moved towards each other. As described herein, that movement causes deflection of the intermediate portion 130 of the pressure element 120 away from the actuation axis 112 that is, in the depicted embodiment, aligned with the elongated body 151 of the actuator 150.

The complete disclosure of the patents, patent documents, and publications identified herein are incorporated by reference in their entirety as if each were individually incorporated. To the extent there is a conflict or discrepancy between this document and the disclosure in any such incorporated document, this document will control.

Illustrative embodiments of the tissue compression devices or methods are discussed herein and some possible variations have been described. These and other variations and modifications in the disclosure will be apparent to those skilled in the art without departing from the scope of the disclosure, and it should be understood that this disclosure is not limited to the illustrative embodiments set forth herein. Accordingly, the disclosure is to be limited only by the claims provided below and equivalents thereof. It should also be understood that this disclosure also may be suitably practiced in the absence of any element not specifically disclosed as necessary herein.

What is claimed is:

1. A tissue compression device comprising:
a pressure element comprising a first end portion, a second end portion, and an intermediate portion located between the first and second end portions; and
retention structure attached to the pressure element, wherein the retention structure is configured to retain the intermediate portion of the pressure element over selected tissue in a selected location; and
wherein the pressure element is configured such that movement of the first and second end portions of the pressure element towards each other along an actuation axis causes the intermediate portion of the pressure element to deflect away from the actuation axis towards the selected tissue.

2. The device of claim 1, further comprising an actuator attached to the first and second end portions of the pressure element, wherein the actuator is configured to move the first and second end portions of the pressure element towards each other.

3. The device of claim 2, wherein the actuator comprises a threaded rod located along the actuation axis and connected to the first and second end portions of the pressure element in a manner configured to move the first and second end portions of the pressure element towards each other when the threaded rod is rotated about the actuation axis.

4. The device of claim 2, wherein the actuator comprises a ratcheting device attached to the first and second end portions of the pressure element.

5. The device of claim 1, wherein the pressure element is configured such that deflection of the intermediate portion away from the actuation axis increases as the first and second end portions are moved closer to each other along the actuation axis, and wherein deflection of the intermediate portion away from the actuation axis decreases as the first and second end portions are moved away from each other along the actuation axis after having been moved closer to each other along the actuation axis.

6. The device of claim 1, wherein the intermediate portion of the pressure element comprises a substantially flat surface facing away from the actuation axis.

7. The device of claim 1, wherein the pressure element further comprises a conformable pad positioned over the selected tissue.

8. The device of claim 7, wherein the conformable pad comprises a fluid-filled bladder.

9. The device of claim 1, wherein the intermediate portion of the pressure element is configured to allow visual access to the selected tissue when the intermediate portion contacts the selected tissue.

10. A method comprising:
positioning the intermediate portion of a pressure element of a tissue compression device over selected tissue at a selection location on a patient, wherein the intermediate portion of the pressure element is located between a first end portion and a second end portion of the pressure element; and
moving the first and second end portions of the pressure element closer together along an actuation axis while retaining the intermediate portion of the pressure element over the selected tissue, wherein the intermediate portion applies increasing pressure on the selected tissue when the first and second end portions of the pressure element are moved closer to each other, and wherein the increasing pressure is directed away from the actuation axis.

11. The method of claim 10, wherein the selected tissue is located on a limb of a patient, and wherein the tissue compression device encircles the limb, and further wherein the actuation axis is transverse to a limb axis extending along a length of the limb.

12. The method of claim 10, wherein the tissue compression device comprises an actuator that comprises a threaded rod attached to the first and second end portions of the pressure element, wherein the threaded rod is located along the actuation axis, wherein moving the first and second end portions of the pressure element closer together comprises rotating the threaded rod in a first direction about the actuation axis.

13. The method of claim 12, further comprising moving the first and second end portions of the pressure element away from each other by rotating the threaded rod in a second direction about the actuation axis.

14. The method of claim 10, wherein the method further comprises attaching the tissue compression device to a patient such that the intermediate portion of the pressure element is positioned over the selected tissue, wherein the attaching comprises:
attaching a first end of a retention structure to the first end portion of the pressure element;
wrapping the retention structure around a limb on which the selected tissue is located; and
attaching a second end of the retention structure to the second end portion of the pressure element.

15. A tissue compression device comprising:
a pressure element comprising a first end portion, a second end portion, and an intermediate portion located between the first and second end portions; and
retention structure attached to the pressure element, wherein the retention structure is configured to retain an intermediate portion of the pressure element over selected tissue in a selected location on a limb by forming a closed loop encircling the limb, wherein the encircled limb defines a limb axis extending through the closed loop;
wherein the pressure element is configured such that the intermediate portion of the pressure element deflects in a first direction towards the limb as the first and second end portions of the pressure element move towards each other in a second direction, wherein the first direction is transverse to both the second direction and the limb axis.

16. The device of claim 15, further comprising an actuator attached to the first and second end portions of the pressure element, wherein the actuator is configured to move the first and second end portions of the pressure element towards each other in the second direction.

17. The device of claim 16, wherein the actuator comprises a threaded rod aligned with the second direction and connected to the first and second end portions of the pressure element in a manner configured to move the first and second end portions of the pressure element towards each other when the threaded rod is rotated about an actuation axis extending along a length of the threaded rod.

18. The device of claim 16, wherein the actuator comprises a ratcheting device attached to the first and second end portions of the pressure element.

19. The device of claim 15, wherein the pressure element is configured such that deflection of the intermediate portion in the first direction towards the limb increases as the first and second end portions are moved closer to each other along the second direction, and wherein deflection of the intermediate portion in the first direction decreases as the first and second end portions are moved away from each after having been moved closer to each other along the second direction.

20. The device of claim 15, wherein the intermediate portion of the pressure element comprises a substantially flat surface over the selected tissue in the selected location on the limb when the tissue compression device encircles the limb.

21. The device of claim 15, wherein the pressure element further comprises a conformable pad positioned over the selected tissue.

22. The device of claim 21, wherein the conformable pad comprises a fluid-filled bladder.

23. The device of claim 15, wherein the intermediate portion of the pressure element is configured to allow visual access to the selected tissue when the intermediate portion is positioned over the selected tissue.

24. A method comprising:
positioning the intermediate portion of a pressure element of a tissue compression device over selected tissue on a limb such that the tissue compression device encircles the limb and wherein the limb comprises a limb axis that extends through an opening formed by the tissue compression device, wherein the intermediate portion of the pressure element is located between a first end portion and a second end portion of the pressure element; and
moving the first and second end portions of the pressure element closer together along a first direction while retaining the intermediate portion of the pressure element over the selected tissue, wherein the intermediate portion moves in a second direction to apply increasing pressure on the selected tissue when the first and second end portions of the pressure element are moved closer to each other, and wherein the first direction is transverse to both the second direction and the limb axis.

25. The method of claim 24, wherein the tissue compression device comprises an actuator that comprises a threaded rod attached to the first and second end portions of the pressure element, wherein the threaded rod is aligned with the first direction, wherein moving the first and second end portions of the pressure element closer together along the first direction comprises rotating the threaded rod about the actuation axis.
